(19) United States
(12) Patent Application Publication
(10) Pub. No.: US 2013/0123934 A1
(43) Pub. Date: May 16, 2013

(54) GROOVED PANCREATIC STENT

(71) Applicant: Riad Azar, New York, NY (US)

(72) Inventor: Riad Azar, New York, NY (US)

(21) Appl. No.: 13/468,586

(22) Filed: May 10, 2012

Related U.S. Application Data
(60) Provisional application No. 61/485,985, filed on May 13, 2011.

(51) Int. Cl. A61F 2/04

(52) U.S. Cl. CPC A61F 2/04
USPC 623/23.7; 29/592; 29/428

(57) ABSTRACT

The invention generally relates to pancreatic devices and methods for guiding cannulation of the bile duct. In certain aspects, the invention provides a tubular stent comprising groove along the length of at least a portion of the stent. In certain aspects, the invention provides a method for cannulating a bile duct.

### CROSS-REFERENCE TO RELATED APPLICATION

This application claims the benefit of U.S. Provisional Application No. 61/485,985, filed May 13, 2011, the contents of which are herein incorporated by reference in their entirety.

### FIELD OF THE INVENTION

The present invention relates to pancreatic devices and methods for guiding cannulation of the bile duct.

### BACKGROUND

Endoscopic Retrograde Cholangiopancreatography (ERCP) is an endoscopic procedure used to access the bile duct. Cannulation of the bile duct can be problematic, given the close proximity of the bile duct and the pancreatic duct. Often, when trying to cannulate the bile duct (i.e., place a catheter in the bile duct), the instrument ends up in the pancreatic duct, which leads to an increased risk of pancreatitis in the patient. Difficulties in bile duct cannulation occurs in overt 20% to 30% of ERC procedures. Currently available pancreatic stents are not designed to specifically facilitate cannulation of the bile duct. In fact, the currently available pancreatic stents occlude the papillary orifice (i.e., where the bile duct and the pancreatic duct merge). As such, there exists a need for an improved pancreatic stent which does not occlude the papilla and thereby facilitates access to the bile duct in difficult bile duct cannulation procedures.

### SUMMARY

The invention provides a stent that has a groove allowing for cannulation of a duct without blockage by tissue. A groove can be positioned on a surface of a stent longitudinally, such that a longest dimension of the groove is parallel to a predominant axis of the stent. The groove can be positioned to correspond to a location of the papilla when the stent is inserted into the pancreatic duct.

In certain aspects, the invention provides a tubular stent comprising groove along the length of at least a portion of the stent. The stent may include a hollow portion at either or both of a proximal end and distal end. Each hollow portion can optionally include a main hole, allowing a guide wire to pass through the hollow portions while passing along the outside of the stent at a non-hollow portion. A hollow portion can further include one or a number of opening holes, for example, for drainage.

In certain aspects, the invention provides a method of cannulating a bile duct. Methods of the invention include cannulating the pancreatic duct of a subject with a first stent comprising a non-hollow groove disposed along the length of at least a portion of the stent such that the non-hollow grooved portion is disposed proximal to the papilla of the subject and cannulating the bile duct with a second stent by inserting the second stent along the grooved portion of the first stent to guide the second stent into the bile duct.

In certain aspects, the invention provides a method of making a stent, comprising obtaining a tubular material having a length and an open lumen along at least a portion of the length of the tubular material and adding a groove along a surface. The groove may be provided by creating an open channel along an outer surface of the stent. Preferably, the groove is less than about 5 mm long. Methods further include making one or more main hole through a surface of the tubular material into the lumen and optionally inserting a guide wire through the hole and through the lumen. Further, one or any number of drainage holes can be added at any portion of the tubular material.

### BRIEF DESCRIPTION OF THE DRAWINGS

In the drawings, In the drawings, like structures and items typically are referenced by the same or similar reference numbers throughout the various views. The illustrations in the drawings are not necessarily drawn to scale, the emphasis instead being placed generally on illustrating the principles of the invention and the disclosed embodiments.

FIG. 1 illustrates organs of a human body.

FIG. 2 includes a cutaway view of a pancreas and duodenum.

FIG. 3 depicts the anatomy of the pancreatic duct, the bile duct and the papillary orifice.

FIG. 4 depicts an exemplary embodiment of a pancreatic duct cannulation in which the papillary orifice is not blocked.

FIG. 5 depicts a blocked papillary orifice.

FIG. 6 shows a stent according to certain embodiments of the invention.

FIG. 7A depicts an exemplary embodiment of a grooved pancreatic stent according to the invention, the groove facilitating access to the bile duct through the papillary orifice when the pancreatic duct is cannulated; and

FIG. 7B is a cross section of the grooved portion of the stent in the papilla as depicted in FIG. 7A.

FIG. 8A depicts an exemplary embodiment of a grooved pancreatic stent according to the invention;

FIG. 8B shows a cross section of stent 403 along line B-B′ shown in FIG. 8A;

FIG. 8C shows a cross section of stent 403 along line C-C′ shown in FIG. 8A; and

FIG. 8D shows a cross section of stent 403 along line D-D′ shown in FIG. 8A.

FIG. 9 is a cut-away view of a stent according to certain embodiments.

FIG. 10 depicts a stent with a guide wire inserted.

FIG. 11 depicts a stent partially inserted into the pancreatic duct.

FIG. 12 depicts an exemplary embodiment of a grooved portion of a pancreatic stent according to the invention, shown in cross section.

FIG. 13 depicts the grooved portion depicted in FIG. 12 disposed in a papillary orifice, shown in cross section.

FIG. 14 depicts an alternative embodiment of a grooved portion of a pancreatic stent with a lumen according to certain embodiments, shown in cross section.

FIG. 15 depicts the grooved portion depicted in FIG. 14 disposed in a papillary orifice, shown in cross section.

### DETAILED DESCRIPTION

The present invention provides improved pancreatic stents and methods for facilitating cannulation of the bile duct using the pancreatic stents of the invention. The pancreatic duct is a duct that joins the pancreas to the common bile duct to supply pancreatic juices which aid in digestion.

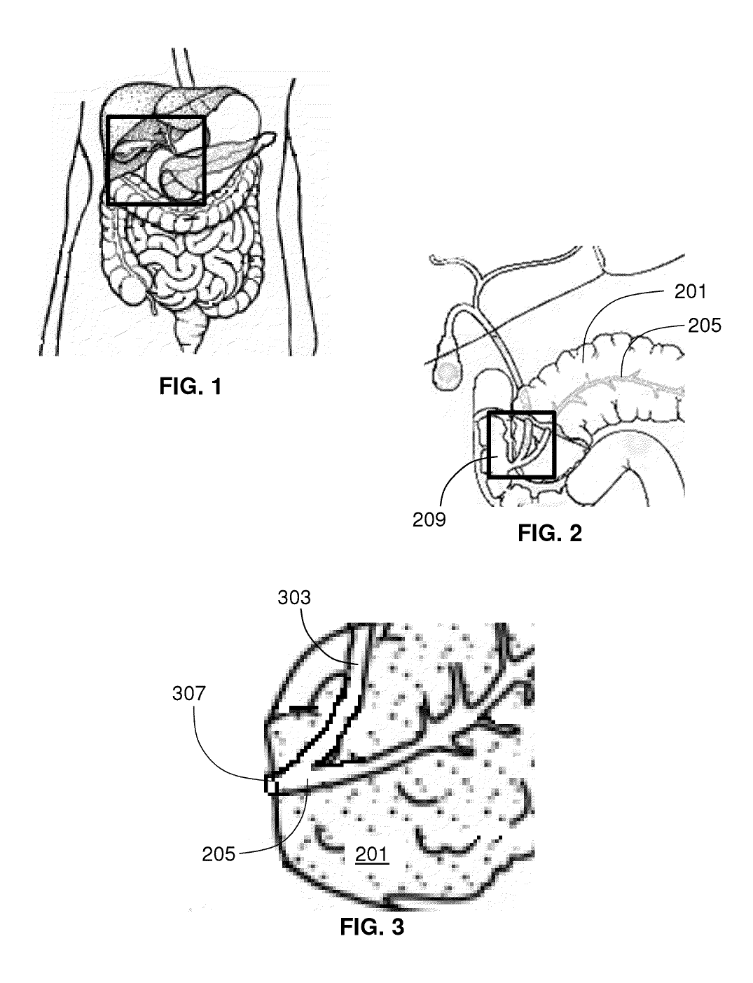

FIG. 1 illustrates organs of a human body. FIG. 2 includes a cutaway view of pancreas 201 and duodenum 209. Pancreatic duct 205 can be seen extending through pancreas 201. Pancreatic duct 205 joins the common bile duct 303 just prior to the ampulla of Vater, after which both ducts perforate the medial side of the second portion of duodenum 209 at the major duodenal papilla 307. (see FIGS. 2 and 3). In instances where a surgeon is having difficulty accessing the bile duct, the surgeon may choose to cannulate the pancreatic duct with a stent. After this, physician controlled wire-guided cannulation of the bile duct can be achieved by using the pancreatic stent to direct a sphincterotome into the biliary duct orifice. Cannulation of the pancreatic duct with a stent also helps to reduce the risk of post-ERCP pancreatitis.

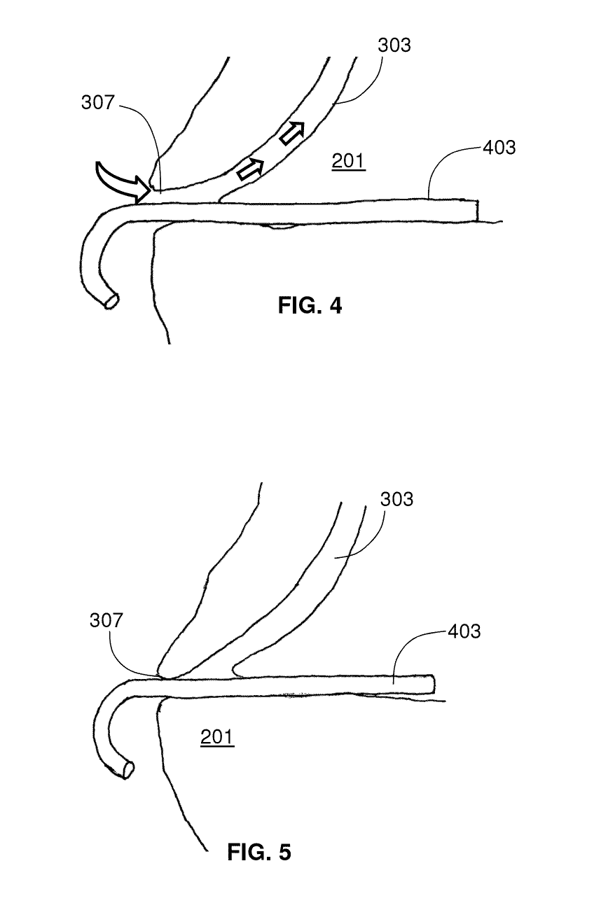

Placing a stent in the pancreatic duct serves to block the pancreatic duct and thereby help to guide cannulation of the bile duct. Once pancreatic duct 205 is cannulated with a stent, physician controlled wire-guided cannulation can attempted by using the pancreatic duct stent to direct a sphincterotome into the bile duct orifice. This procedure can be successful if the papillary orifice is not tight/small (see FIG. 4). However, if the papilla 307 is tight/small, the pancreatic duct stent 403 can block papilla 307 making it difficult for the surgeon to advance anything into the bile duct (see FIG. 5).

The present invention solves this problem by providing grooved pancreatic stents for use in difficult bile duct cannulation procedures which allows access to the bile duct regardless of the size of the papillary orifice. Positioning and use of devices is discussed in U.S. Pat. No. 5,876,450; U.S. Pub. 2010/0114325; U.S. Pub. 2009/0264808; and U.S. Pub. 2009/0048654, the contents of which are hereby incorporated by reference for all purposes.

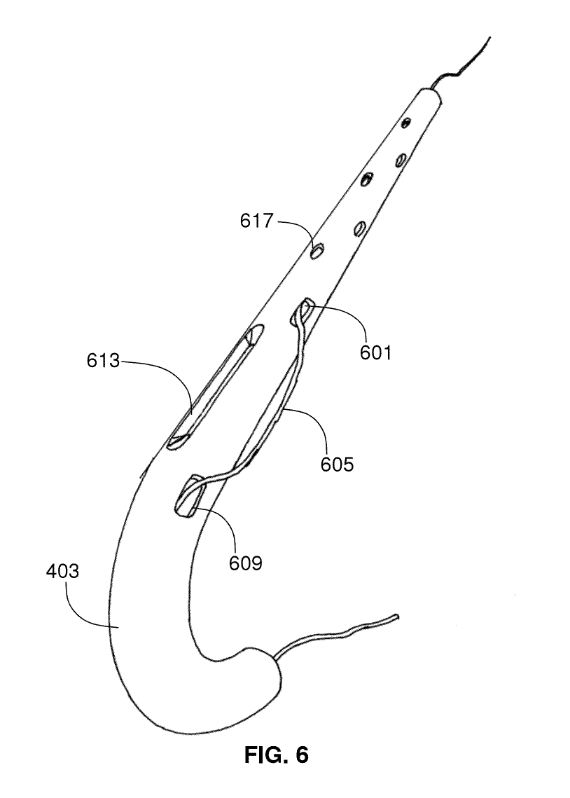

FIG. 6 illustrates a stent 403 according to certain embodiments of the invention. As can be seen in FIG. 6, stent 403 can include either or both of proximal main hole 609 and distal main hole 601 to accommodate guide wire 605. Stent 403 includes at least one groove 613, having any suitable length, width, or depth. In certain embodiments, a length of groove 613 is limited to less than about 50% of an overall length of stent 403 (e.g., a length of groove 513 can be less than about 33% of an overall length of stent 403). Stent 403 may optionally include one or more of opening hole 617. Opening holes are discussed in U.S. Pub. 2010/0114325, the contents of which are hereby incorporated by reference for all purposes. Groove 613 preferably comprises an elongate depression on an external surface of stent 403. Groove 613 may be longitudinal in that a longest dimension of groove 613 is substantially parallel to an axis of a predominant straight portion of stent 403. In some embodiments, groove 613 is not precisely longitudinal. For example, groove 613 may exhibit a rifled orientation, tracing a spiral around a portion of stent 403. Stents are discussed further in U.S. Pub. 2006/0265051, the contents of which are incorporated by reference in their entirety. In certain embodiments, limiting a length of groove 613 according to disclosure herein provides a beneficial result of ease of cannulation of the bile duct. Without being limited by any theory, it is suggested here that the termini of groove 613 aid in positioning any cannula or device located therein. In certain embodiments, stent 403 includes a groove with a terminus at each end along a surface of the stent. That is, the groove is shorter than an overall length of the stent.

Stent 403 is generally shown in figures herein having a “candy-cane” or “shepherd's crook” morphology comprising a straight portion having a distal end and a proximal region, in which the proximal region of the straight portion extends into a curved portion. Stent 403 may comprise a soft, malleable (e.g., elastic, plastic, or a combination thereof) material, and therefore may not exhibit any given depicted form under various conditions. Other morphologies are possible including a stent that is entirety straight, substantially straight, or a stent that has a gentle curve in any straight portion and a proximal end of stent 403 may exhibit forms other than curved including a loop or straight. Stents are depicted in U.S. Pub. 2008/0091275, the contents of which are incorporated by reference for all purposes.

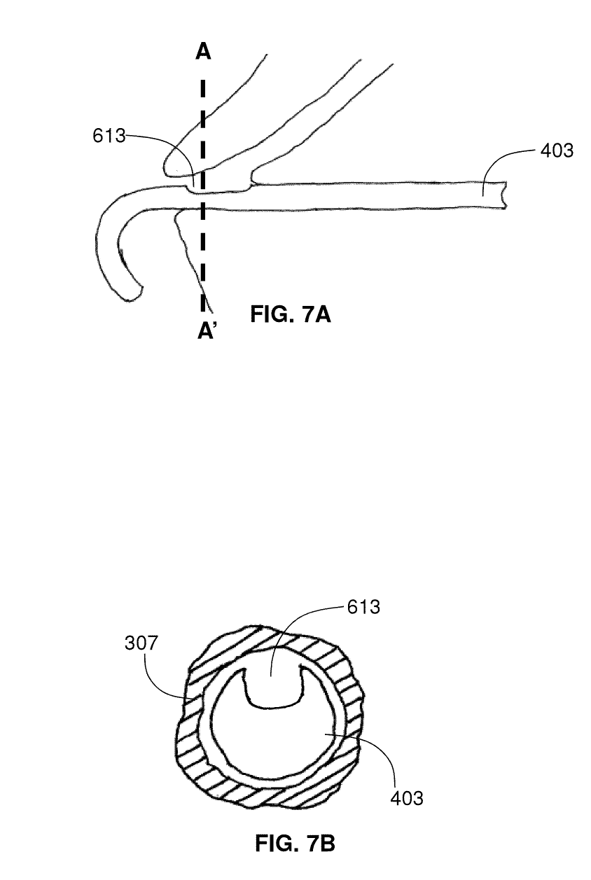

By providing a non-hollow and/or open groove in a pancreatic stent, a space is created between pancreatic stent 403 and the lip/edge of papilla 307 to allow passage of a wire between the stent and papilla 307 to cannulate the bile duct (see FIG. 7A and 7B). As shown in FIG. 7A, stent 403 allows communication among ducts and lumen of tissue. In certain embodiments, groove 613 both enables good fluid communication between a bile duct and a duodenum while also aiding in cannulation.

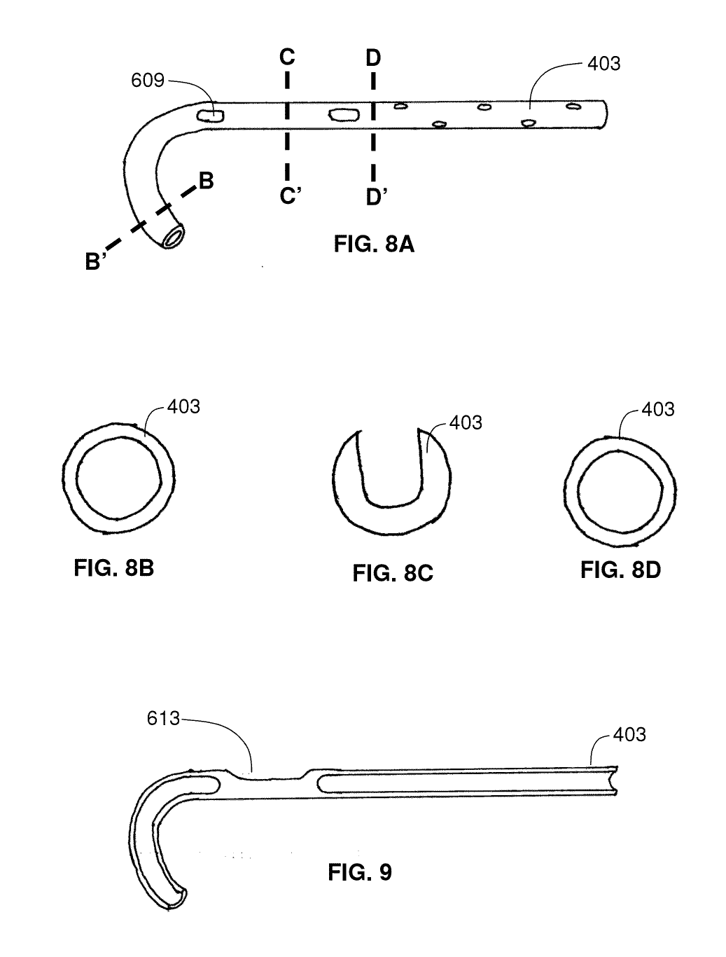

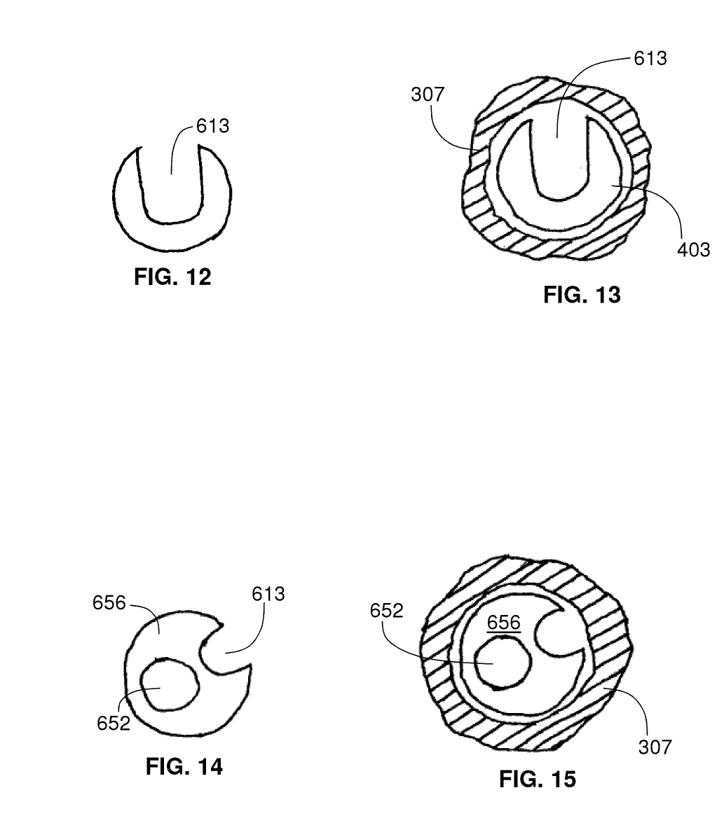

An exemplary embodiment of a pancreatic stent according to the invention is depicted in FIG. 8A. As shown in FIG. 8A, the pancreatic stent contains a non-hollow and/or open grooved portion having a proximal and a distal end. As used herein, the term “proximal” refers to the end of the stent closest to a medical professional when placing the stent in a patient, and the term “distal” refers to the end of the stent farthest away from a medical professional when placing the stent in the patient. The grooved portion can be a U-shaped channel 613 (see e.g., FIGS. 12 and 13), or a tubular member defining a hollow interior channel and containing a single U-shaped or open/grooved channel 613 extending along the outer portion of the tubular wall (see e.g., FIGS. 14 and 15). Discussion of stents and their materials and methods of use can be found in U.S. Pat. No. 7,338,530, the contents of which are hereby incorporated by reference in their entirety for all purposes. FIG. 8B is a cross section of stent 403 along line B-B′ shown in FIG. 8A. FIG. 8C shows a cross section of stent 403 along line C-C′ shown in FIG. 8A. FIG. 8D shows a cross section of stent 403 along line D-D′ shown in FIG. 8A.

As shown in FIG. 9, groove 613 can be disposed substantially between two hollow segments of stent 403. More specifically, the proximal end of the grooved portion is coupled to the distal end of a proximal hollow elongate member. The proximal hollow elongate member may be generally straight, or may include one or more bends along a longitudinal axis of the elongate member. The distal end of the grooved portion is coupled to the proximal end of a distal hollow elongate member. The distal hollow elongate member is generally straight for insertion into the pancreatic duct.

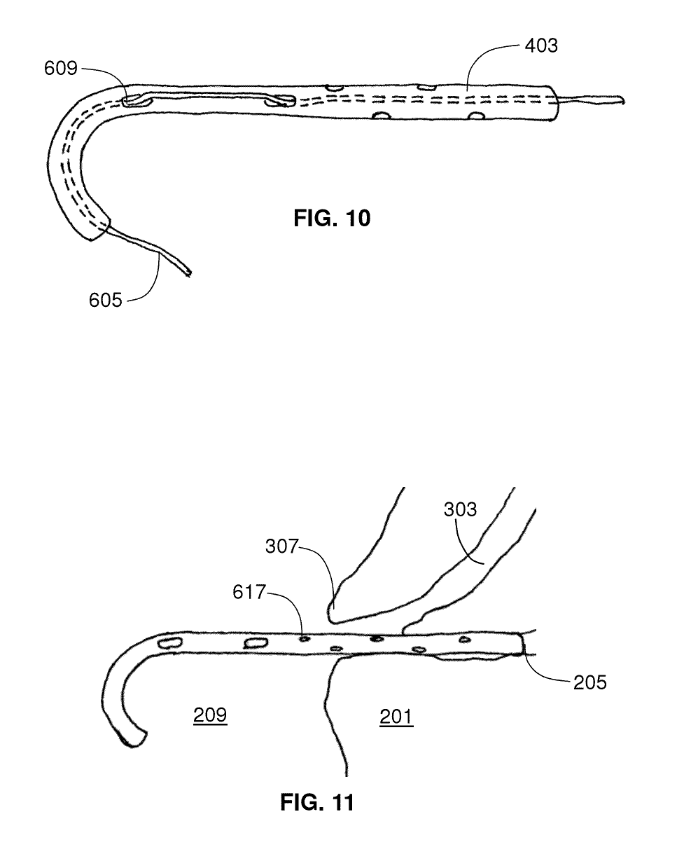

As shown in FIG. 10, the proximal hollow elongate member includes proximal main hole 609, while the distal hollow elongate member includes distal main hole 601 (see also FIG. 6). These main holes are shaped and sized such that guide wire 605 can be disposed through each of them. In this arrangement, the guide wire saddles the grooved, non-hollow portion of the stent.

As shown in FIG. 10, the distal hollow elongate member of the stent is inserted over guide wire 605 until it reaches distal main hole 601 disposed adjacent the grooved portion. Guide wire 605 is threaded through distal main hole 601 such that the wire saddles the grooved, non-hollow portion of the stent until the wire reaches proximal main hole 609 in the proximal, hollow elongate member and is there threaded into the proximal, hollow elongate member. In addition to the main holes, the proximal and distal hollow elongate members optionally include one or more of side hole 617 for drainage of the pancreas 201 into the intestine 209.

As shown in FIG. 11, in certain embodiments, the proximal and distal elongate members include a plurality of side hole 617 for drainage of the pancreas into the intestine. After using the pancreatic stent to facilitate cannulation of the bile duct, the pancreatic stent is pulled partially out of the pancreatic duct to allow drainage of the pancreas into the intestine (FIG. 11).

The grooved portion of the pancreatic stents of the invention is preferable 0.5 mm to 4 mm in length, preferably 0.75 to 3 mm in length, more preferably 1 to 2 mm in length, or any specific value between said ranges. The proximal hollow elongate member and distal hollow elongate member can be the same size. For example, the proximal hollow elongate member and distal hollow elongate member can both be a 3-French, a 4-French, a 5-French, a 6-French, a 7-French, an 8-French, a 9-French sheath or a 10-French sheath. The skilled artisan will recognize that the French scale or French gauge system (sometimes abbreviated as Fr, but also often abbreviated as FR or F) is commonly used to measure the size (diameter) of a catheter (1 Fr=0.33 mm, therefore D (mm)=Fr/3 or Fr=D (mm)×3). Alternatively, the proximal hollow elongate member and the distal hollow elongate member can be different sizes. For example, the proximal hollow elongate member can be larger than the distal hollow elongate member (e.g., 7-French and 5-French, respectively).

Stent 403 can include one or more of groove 613 having any suitable dimensions and position. For example, as shown in FIG. 12, groove 613 can be deep to generously accommodate a cannula or wire. FIG. 13 shows stent 403 in papilla 307 to illustrate the accommodation of groove 613.

As shown in FIG. 14, in certain embodiments, the invention provides an open stent 656 that includes lumen 652 as well as one or more of groove 613 (either of which can accommodate, for example, a guide wire 605). FIG. 15 illustrates stent 656 including lumen 652, in cross-section, inside of papilla 307. Stents with lumen are discussed in U.S. Pat. No. 6,929,664, the contents of which are hereby incorporated by reference for all purposes.

In some embodiments, the pancreatic stents of the invention include additional features such as an external pigtail, one or more internal flanges, one or more external flanges, or any combination thereof.

In certain aspects, the invention provides methods for making or using a stent.

The pancreatic stents of the invention are preferably made of a flexible material, such as a plastic or other pliable, flexible polymer material, that is suitable for internal use and can withstand (i.e., will not degrade or decompose in the presence of) biliary and pancreatic secretions. In certain embodiments, a stent is made from a tubular material. Exemplary materials for the stents of the invention include, but are not limited to the following: the compound sold under the trademark SOFFLEX by Cook Urological Inc. (Spencer, Ind.), a type of polyether urethane, silicone, block co-polymers, urethanes, polyethylene, polystyrene, polytetrafluoroethylene (PTFE), FEP and the like and combinations thereof. In some embodiments, the stents of the invention may be formed from biodegradable materials. A number of bioabsorbable homopolymers, copolymers, or blends of bioabsorbable polymers are known in the medical arts. These include, but are not necessarily limited to, polyesters including poly-alpha hydroxy and poly-beta hydroxy polyesters, polycaprolactone, polyglycolic acid, polyether-esters, poly(p-dioxanone), polyoxaesters; polyphosphazenes; polyanhydrides; polycarbonates including polytrimethylene carbonate and poly(iminocarbonate); polyesteramides; polyurethanes; polyisocyantes; polyphosphazines; polyethers including polyglycols polyorthoesters; expoxy polymers including polyethylene oxide; polysaccharides including cellulose, chitin, dextran, starch, hydroxyethyl starch, polygluconate, hyaluronic acid; polyamides including polyamino acids, polyester-amides, polyglutamic acid, poly-lysine, gelatin, fibrin, fibrinogen, casein, collagen. In certain embodiments, the pancreatic stents of the invention can be made from an alloy material having shape memory and/or superelastic characteristics, such as a nickel-titanium alloy (e.g., nitinol). In certain embodiments, the pancreatic stents of the invention may be constructed from absorbable materials including polylactic acid/polylactide (PLA), polydimethylsiloxane (PDMS), polyglycolide/polyglycolic acid (PGA), and the like. Polymers for use as a material of a stent are discussed in U.S. Pub. 2010/0100170, the contents of which are hereby incorporated by reference for all purposes.

A stent can be made according to methods of the invention by adding a groove to a surface of a tubular material (e.g., by channeling or routing out material with a scalpel or tool, or stripping material from along a length of a surface with a tool). Additionally, currently available tubular stents can be modified to include a grooved, non-hollow portion, to facilitate cannulation of the bile duct by providing access to the papillary orifice, as described herein. Exemplary tubular stents include, but are not limited to, the biliary stent sold under the trademark ST-2 SOEHENDRA TANNENBAUM by Cook Medical Inc (Bloomington, Ind.), the biliary stent sold under the trademark COTTON-LEUNG by Cook Medical, the biliary stent sold under the trademark COTTON-HUIBREGTSE by Cook Medical, the pancreatic stent sold under the trademark GEENEN by Cook Medical, any of the pancreatic wedge stents sold under the trademark JOHLIN by Cook Medical, or the pancreatic stent sold under the trademark ZIMMON by Cook Medical.

Methods include adding proximal main hole 609 or distal main hole 601 by, for example, piercing or drilling with a tool and optionally threading a guide wire 605 through the stent, e.g., using the main holes. For drainage, one or any number of opening holes 617 may optionally be punched or pierced through the stent or tubular material.

The pancreatic stents of the invention can be coated with a therapeutic agent. Alternatively the stents of the invention can elute a therapeutic agent. Examples of therapeutic agents include, without limitation, an antibiotic, a bile deactivating agent, an intestinal brake inducing agent, a hunger suppressing agent.

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### Equivalents

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A stent comprising:
a substantially tubular body; and
a groove along a surface of the stent.

2. The stent of claim 1, wherein the groove is disposed between a proximal hollow segment and a distal hollow segment of the stent, wherein each hollow segment has an aperture at an end of the stent.

3. The stent of claim 2, wherein each of the proximal hollow segment and the distal hollow segment comprises a hole dimensioned for inserting a guide wire.

4. The stent of claim 1, wherein the distal hollow segment comprises a plurality of holes disposed along a length of the distal hollow segment for facilitating drainage of a fluid.

5. The stent of claim 1, wherein the groove is less than about 5 mm long.

6. A stent comprising:
a first elongate member having a proximal and a distal end and comprising a hollow lumen;
a second elongate member comprising at least one groove on an outer surface of the member extending along the length of the member, the second member comprising a proximal end and a distal end; and
a third elongate member having a proximal and a distal end and comprising a hollow lumen;
wherein the proximal end of the grooved member is coupled to the distal end of the first elongate member, and the distal end of the grooved member is coupled to the proximal end of the third elongate member.

7. The stent of claim 5, wherein the second member comprises a U-shaped channel.

8. The stent of claim 5, wherein the second member comprises a hollow shaft having at least one grooved channel disposed along the length of the outer surface of the shaft.

9. The stent of claim 5, wherein the first and third elongate members each comprise a hole adjacent to the second member, the holes being dimensioned for sliding a guide wire therethrough.

10. The stent of claim 5, wherein the first and third elongate members each comprise a plurality of holes disposed along the length of the elongate members for facilitating drainage of a fluid from the stent.

11. The stent of claim 5, wherein the first elongate member comprises at least one bend along the length of the member.

12. The stent of claim 5, wherein the first and third elongate members comprise the same size.

13. The stent of claim 11, wherein the first and third elongate members are 5-French or 7-French segments.

14. The stent of claim 5, wherein the first and third elongate members are different sizes.

15. The stent of claim 13, wherein the first elongate member is a 7-French segment and the third elongate member is a 5-French segment.

16. The stent of claim 5, wherein the second elongate member is 1-2 mm in length.

17. A method of cannulating the bile duct in a subject in need thereof, said method comprising the steps of:
cannulating the pancreatic duct of the subject with a first stent comprising a non-hollow groove disposed along the length of at least a portion of the stent such that the non-hollow grooved portion is disposed proximal to the papilla of the subject;
cannulating the bile duct with a second stent by inserting the second stent along the grooved portion of the first stent to guide the second stent into the bile duct.

18. A method of making a stent, comprising:
obtaining a tubular material having a length and an open lumen along at least a portion of the length of the tubular material; and
adding a groove along a surface of the tubular material.

19. The method of claim 18, wherein the groove comprises an open channel along an outer surface of the tubular material, and a length of the groove is less than 5 mm.

20. The method of claim 18, further comprising making a hole a through a surface of the tubular material into the lumen.

21. The method of claim 20, further comprising inserting a guide wire through the hole and through the lumen.
